(19) United States
(12) Patent Application Publication
(10) Pub. No.: US 2002/0162972 A1
(43) Pub. Date: Nov. 7, 2002

(54) Stationary enclosure for sterilizing stethoscopes using ultraviolet light

(76) Inventor: Lawrence Pleet, Bell Canyon, CA (US)

Correspondence Address: LAWRENCE PLEET, M.D., 9 FLINTLOCK LANE, BELL CANYON, CA, 91307, US

(21) Appl. No.: 10/117,956

(22) Filed: Apr. 8, 2002

Related U.S. Application Data
(60) Provisional application No. 60/284,132, filed on Apr. 17, 2001.

(51) Int. Cl. G21G 5/00

(52) U.S. Cl. 250/492.1

(57) ABSTRACT

An enclosure is provided which enables sterilization of all or part of a single or multiple stethoscopes or other items commonly re-used in the care of medical patients, such as a blood pressure cuff, EKG leads, or pulse oximeter probe. The enclosure is designed to easily suspend the stethoscope or other items in a manner which allows complete exposure of all areas to ultraviolet light. The enclosure is further designed to prevent excape of the ultraviolet light into the external environment when the device is active. A manual or predetermined timing device insures that sufficient time of exposure to kill all potential contaminating agents by means of intense ultraviolet light radiation will occur.

### NON-PROVISIONAL PATENT APPLICATION APR. 4, 2002

Priority status is claimed under application number 60/284,132; Filing date Apr. 17, 2001 Confirmation No. 6051, Titled “Stationery Enclosure for Sterilizing Stethoscope Earpiece and Bell Using Ultraviolet Light”.

STATIONARY ENCLOSURE FOR STERILIZING STETHOSCOPES OR OTHER MEDICALLY RE-USABLE OBJECTS USING ULTRA-VIOLET LIGHT

SOLE INVENTOR: Lawrence Pleet, M.D., 9 Flintlock Lane, Bell Canyon, Calif. 91307-1128

FIELD OF SEARCH: 250/455.11,454.11,493.1,504R 422/24,300

IPC Class: A61B July 2002

CLASS: 250/455.11; 181/131; 250/504.R

REFERENCES SITED: U.S. PATENT DOCUMENTS 5892233 January 1996 Clement 250/455.11 5641464 June 1997 Briggs et. al.

### PRIOR ART

The prior art consists of U.S. Pat. No. 5,892,233 which is a device which simultaneously stores and sterilizes the head of a stethoscope within the pocket of a user during periods of non usage. This device only sterilizes the head and does not address the problem of remaining components of the stethoscope. There is no prior art regarding a stationary enclosure for enclosing a stethoscope or other medically related items, which stationary enclosure can be conveniently used in medical offices, emergency rooms at hospitals, hospital wards, nursing stations, operating rooms, and intensive care units. Many personnel have their own stethoscopes which they carry with them, but which could be placed within the described invention for a brief interval of time and thus avoid the possibility of transferring infectious disease to unsuspecting patients. Hospital often supply stethoscopes in convenient areas where they may be suspended haphazardly from medicine carts, coat hangers, and the like. U.S. Pat. No. 5,641,464 describes a device which cleans the head of a stethoscope using an enclosure which contains a cannister which releases an antimicrobial spray upon activation of a manual lever mechanism. This is not a user friendly device and ignores other components of the stethoscope which may be contaminated.

### BRIEF DESCRIPTION OF THE DRAWINGS

The accompanying drawings illustrate the invention. In such drawings:

FIG.1 is a fragmented and schematic perspective view illustrating a partially open enclosure embodying the novel features of the invention.

FIG. 2 is a schematic illustration of the invention without the hinged front cover and showing the stethoscope mounted on rods and U-shaped dependent support.

### DETAILED DESCRIPTION OF THE PREFERRED EMBODIMENTS

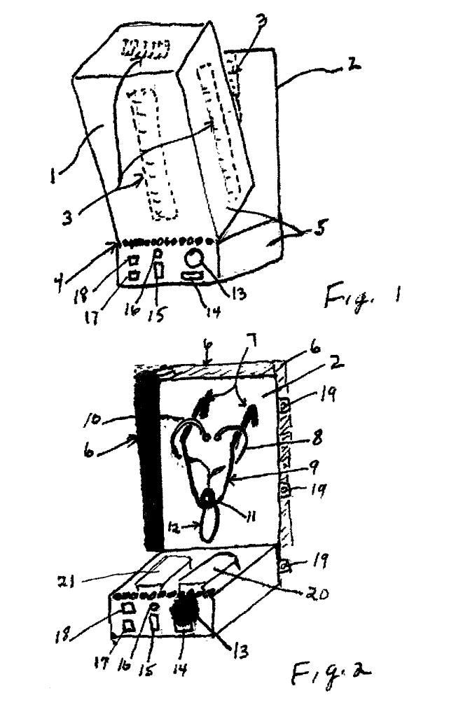

As shown in the exemplary drawings, an enclosure referred to generally in FIG. 1 by the reference numeral 5 is provided for enclosing one or more stethoscopes or other medically related objects for the purpose of subjecting said objects to intense ultraviolet light for the purpose of sterilization. FIG. 1 and FIG. 2 show the enclosure 5 with a hinge mechanism 4 allowing the front cover 1 to open so as to fully expose the rods 7 upon which the curved metal portions of the stethoscope 10 rest. The rods may be made of a translucent material such as acrylic which can transmit the ultraviolet light. FIG. 2 shows the connecting tubing 12 of the stethoscope making a 180 degree turn allowing the bell-diaphragm 11 portion of the stethoscope to rest on the

U-shaped dependent member 9 which is integral with the rods 7. It is understood that the U shaped dependent member 9 may be omitted from the preferred embodiment and will result in the stethoscope being solely suspended from the rods 7, resulting in an increase in the height of the enclosure. Alternatively, the rods 7 may be connected in the front, forming a U shape. The front portion of the U may itself provide support or may have hooks attached upon which items may be suspended. It is understood that with reference to FIG. 1, the hinged front cover 1 may be hinged in alternate locations to allow the cover 1 to open sideways as in a conventional door opening. It is further understood that the hinged front cover 1 is designed to close so as to prevent ultraviolet light from escaping the enclosure 5. The hinge mechanism 4 will be provided with a microswitch which will provide power to the light sources 3 only with the door closed. FIG. 2 shows a timing mechanism 20 contained in the lower portion of the enclosure. The timing mechanism 20 includes a time set adjusting knob 13 and time readout window 14. FIG. 2 shows a power supply 21 contained in the lower portion of the enclosure and which is integral with an on-off power switch 15 and a power on indicator light 16. FIG. 1 and FIG. 2 show indicator light 17 which when lit indicates that sterilization is in progress. FIG. 1 and FIG. 2 show indicator light 18, which when on indicates that sterilization is complete. FIG. 2 shows the enclosure 5 mounted on the wall 6 by means of mounting tabs 19. It is understood that the enclosure 5 may be self supporting and could be placed on any convenient support such as a table or desk A variety of further modifications and improvements to the enclosure described in the invention will be apparent to those skilled in the art. Accordingly, no limitation on the invention is intended by way of the forgoing description and accompanying drawings, except as set forth in the appended claims.

### SUMMARY

Currently there is no stationary method for sterilizing all or portions of a stethoscope when the stethoscope is not in use. There is also no stationary method for sterilizing other re-usable medically related items such as a blood pressure cuff, EKG leads, or pulse oximeter probes which are in common usage in the operating room environment. The object of the present invention is to provide a stationary enclosure which may be wall mounted and into which the entire stethoscope or related medical items may be placed for rapid and efficient sterilization by means of ultraviolet radiation. Such an enclosure can also be used for non-medical items requiring sterilization such as toys shared by children. Once the enclosure door is closed, the ultraviolet light source is activated and over a predetermined time using a timing device which may be preset, the enclosed items may be sterilized. A further object of the invention is to provide maximum exposure of all portions of the stethoscope or other items to the ultraviolet light source. This is accomplished using high intensity light sources, a polished interior, and translucent suspension means allowing UV light to pass. The object of the invention is to prevent the spread of infectious agents to patients and other individuals caused by the shared usage of stethoscopes and other re-usable medical items as well as non-medical items such as toys. It is noted that those versed in the art may adapt the invention for sterilization of additional items which have the potential for spreading infection.

What is claimed is:

1. A stationary enclosure designed to house all or portions of a single or multiple stethoscopes or other medically related re-usable items, thereby enabling the enclosed items to be sterilized by means of ultraviolet light radiation.

2. The device of claim one which contains one or more ultraviolet light sources.

3. The device of claim one which has dual rod or U-shaped projecting supports in the upper interior portion upon which the curved upper metallic portions of the stethoscope or other related items may be suspended.

4. The devices of claim three which may be composed of translucent material such as acrylic to allow for transmission of ultraviolet light.

5. The device of claim one which has a hinged door which allows easy access to the projecting supports and entire interior.

6. The device of claim five which is designed to prevent the excape of ultraviolet light to the external environment, when the door is in a closed position.

7. The device of claim five which, when open, automatically disconnects the power source to the ultraviolet lights.

8. The device of claim one which can be made of injection molded plastic or stainless steel.

9. The device of claim two which can produce a range of 10,000-46,000 microwatts per second per centimer square.

10. The device of claim three which has an attached U or V shaped plastic or metallic extension attached to and extending below the upper projecting supports upon which the bell-diaphragm of the stethoscope or other items may be suspended.

11. The device of claim four which upon closing activates a microswitch which allows current to flow to the ultraviolet light source and which upon opening causes the current to be interrupted.

12. The device of claim one which may have slotted or perforated tabs projecting outwardly from the rear surface, which may be used to mount the enclosure to a flat surface such as a wall.

13. The device of claim one whose interior is highly polished in order to increase the effectiveness of the ultraviolet light sources.

14. The device of claim one which has a timing device which activates when the door is closed and which can be manually set or preset to the time required to achieve sterilization.

15. The device of claim one which may enclose non-medical items suitable for sterilization by means of ultraviolet light.
